(19) United States
(12) Patent Application Publication
(10) Pub. No.: US 2021/0346577 A1
(43) Pub. Date: Nov. 11, 2021

(54) COATING COMPOSITION FOR MEDICAL IMPLANTS

(71) Applicants: Region Syddanmark, Vejle (DK); Syddansk Universitet, Odense M (DK)

(72) Inventors: Chris Halling DREYER, Slagelse (DK); Ming DING, Odense SV (DK)

(21) Appl. No.: 17/284,335

(22) PCT Filed: Oct. 15, 2019
(86) PCT No.: PCT/EP2019/077866

§ 371 Date: Apr. 9, 2021

(30) Foreign Application Priority Data
Oct. 18, 2018 (EP) 18201249.2

(51) Int. Cl. A61L 31/10; A61L 31/02; A61L 27/34; A61L 27/06; A61L 27/54; A61L 31/16

(52) U.S. Cl. CPC A61L 31/10; A61L 31/022; A61L 27/34; A61L 27/06; A61L 2430/24; A61L 31/16; A61L 2420/02; A61L 2300/414; A61L 2430/02; A61L 27/54

(57) ABSTRACT

The present invention relates to a method for coating medical implants. In particular, the present invention relates to coating compositions comprising PDLLA, VEGF, chloroform, an organic solvent different from chloroform, preferably a carrier such as BSA and water for coating medical implants. Such coated medical implants show improved bone regeneration and ingrowth after implantation.

### TECHNICAL FIELD OF THE INVENTION

The present invention relates to a method for coating medical implants or a part of a medical implant. In particular, the present invention relates to coating compositions comprising PDLLA, VEGF, chloroform, an organic solvent different from chloroform and water for coating medical implants.

### BACKGROUND OF THE INVENTION

Bone regeneration has attracted an increasing interest in the field of orthopaedic research due to increasing elderly population, increasing fracture incidence, and the need for a sustainable and unlimited method to ensure repair and regeneration. The current use in larger bone defects is often allografts, harvested from spare bone after insertion of arthroplasty or from cadavers. However, allografts are associated with risk of disease transmission, immunogenicity and donor site morbidity. Furthermore, the available bone banks cannot keep up with the clinical demand.

To get an alternative to the above listed challenges, different biomaterials have been tried. The theory behind such designs is to enhance critical factors in the bone remodeling process, such as osteogenic and angiogenic stimulation, release methods, time point for stimulation, dosages, costs and clinical applicability of usage.

Blood supply is a common limitation for optimal bone formation, and the chemokine vascular endothelial growth factor (VEGF) is the main stimulator of blood vessels. This chemokine is derived from Mesenchymal stem cells (MSCs) and endothelial cells and induces angiogenesis by increasing endothelial proliferation, migration, vessel permeability, tube formation, and survival.

G. Schmidmaier et al. (Biodegradable Poly(D,L-Lactide) Coating of Implants for Continuous Release of Growth Factors. J Biomed Mater Res. 2001;58(4):449-55) discloses that local application of growth factors like insulin like growth factor-I (IGF-I) and transforming growth factor-beta 1 (TGF-β1) from a biodegradable thin layer of poly(D,L-lactide) (PDLLA) coated implants could stimulate fracture healing.

US 2001/0031274 A1 also discloses that application of growth factors like insulin like growth factor-I (IGF-I) and transforming growth factor-beta 1 (TGF-β1) from a biodegradable thin layer of poly(D,L-lactide) (PDLLA) coated implants may stimulate fracture healing.

Hence, an improved method for coating synthetic medical implants would be advantageous, and in particular a more efficient and/or reliable coating composition would be advantageous.

### SUMMARY OF THE INVENTION

In here a coating composition for medical implants (or parts of medical implants) is disclosed, showing promising results in bone ingrowth, in formation of bone in critical size defects (CSD) in the trabecular bone structure and in theory also antibacterial effects. This can give implication in both normal bone structure but also in patients suffering from avascular necrosis or osteoporotic fracture that have decreased angiogenic and osteogenic properties. All components in the coating of the invention have been approved and administered in humans by the FDA.

Thus, in an embodiment, the present invention relates to a method for coating medical implants. In particular, the present invention relates to coating compositions comprising PDLLA, VEGF, chloroform, an organic solvent different from chloroform, preferably a carrier such as BSA and water for coating medical implants. Such coated medical implants show improved bone regeneration and ingrowth after implantation.

Thus, an object of the present invention relates to the provision of an improved coating composition for medical implants. Examples of improvements may be:
Improved bone ingrowth and regeneration;
Avoidance of allografts or other substitute materials;
Fast coating method;
Slow release of VEGF; and
Antibacterial effects.

In particular, it is an object of the present invention to provide a medical implant with improved bone ingrowth properties. The coating composition according to the present invention preferably comprises poly-DL-lactic acid (PDLLA), chloroform, ethanol and water in combination with vascular endothelial growth factor (VEGF). In example 2, such coated implants are tested in sheep models and performs at least equally well as an allograft in relation to bone ingrowth. Examples 3-6 show further analysis of the coating composition and compares it to the coating composition disclosed in G. Schmidmaier et al.

Thus, one aspect of the invention relates to a method for coating a medical implant (or part of a medical implant), the method comprising
a) providing a medical implant;
b) providing a liquid (coating) composition comprising
   0.01-0.2 mg/μl PLA, preferably poly(DL-lactic) acid (PDLLA);
   0.1-10 ng/μl VEGF;
   30-70% (by volume) chloroform;
   20-50% (by volume) organic solvent (preferably different from chloroform), more preferably an alcohol, even more preferably ethanol; and
   2-10% (by volume) water.
c) coating said medical implant (or part of the medical implant to be coated) in vitro with the composition of step b);
d) drying said coated medical implant; and
e) optionally, repeating step c) to d) at least one time.

Preferably, said liquid composition further comprises a carrier, more preferably the carrier is BSA.

Preferably, said medical implant is selected from the group consisting of a screw, a joint, a fastening mean, a fracture fixation and an endoprosthetic device; and/or preferably said medical implant comprises or consists of metal, preferably titanium, steel or thantalum, pure magnesium and combinations with alloys, plastic, Hydroxyapatite (HA), elastomers, acrylic resins, and ceramics including TCP.

Another aspect of the present invention relates to a liquid (coating) composition comprising
0.01-0.2 mg/μl PLA, preferably poly(DL-lactic) acid (PDLLA);
0.1-5 ng/μl) VEGF;
30-70% (by volume) chloroform;
20-50% (by volume) organic solvent, preferably an alcohol, more preferably ethanol; preferably the organic solvent being different from chloroform;
optionally and preferred a carrier, preferably the carrier is BSA and
2-10% (by volume) water.

Yet another aspect of the present invention relates to the use of a liquid coating composition according to the invention, for coating a medical implant (or part of a medical implant to be coated).

A further aspect relates to a medical implant obtained/obtainable by a method according to the invention.

Yet a further aspect relates to a medical implant (or part of a medical implant) coated on the surface with poly(DL-lactic) acid and VEGF.

Another aspect relates to a kit of parts comprising
A first container comprising PLA, preferably poly(DL-lactic) acid (PDLLA);
A second container comprising VEGF;
A third container comprising chloroform;
A fourth container comprising an organic solvent, preferably an alcohol, more preferably ethanol, preferably the organic solvent being different from chloroform;
optionally, a fifth container comprising water;
optionally and preferably a sixth container comprising a carrier; and
optionally, instructions for preparing a liquid coating composition according to the invention and/or performing a method according to the invention.

Finally, the invention relates to the use of a kit according to the invention, for coating a medical implant (or part of a medical implant). Preferably, said medical implant is selected from the group consisting of a screw, a joint, a fastening mean, a bone filler, a fracture fixation and an endoprosthetic device; and/or said medical implant comprises or consists of metal, preferably titanium, steel or thantalum, pure magnesium and combinations with alloys, plastic, Hydroxyapatite (HA), elastomers, acrylic resins, and ceramics including TCP.

### BRIEF DESCRIPTION OF THE FIGURES

FIG. 1 shows A) An illustration of the titanium implant used in the design and B) the size and measurements. The inner length and outer diameter of the titanium implant are 10mm×10mm and the 2mm concentric gap around the implant resulting in 0.5 mL—the region of interest in the analysis.

FIG. 2 shows the mixture in the method described in Schmidmaier et al., when mixed with the VEGF protein. The VEGF solution is accumulated at the top of the liquid (arrow) and cannot be diluted by rotation or vibration.

FIG. 3 shows A: Illustration of the placement of the 10mm ×10mm titanium implant into the trabecular bone structure in the distal femur condyle. Notice the placement of the implant after a 90° rotation according to the histological images. The implants are then embedded and sectioned. B: Illustration of an implant after 12 weeks of observation with an expected daily release of 100 ng VEGF/day. The grey areas are bone, white areas are either fibrous tissue or bone marrow and black areas are the implant. C-E: Implants coated with different amount of VEGF C: Expected daily release of 500ng VEGF/day. D: Expected daily release of 1000 ng VEGF/day E: Expected daily release of 2000 ng VEGF/day. F: Empty implant without coating or VEGF.

FIG. 4 shows microCT images of the different implants. A: Illustration of the region of interest displayed in the scans, which correlates to the 2 mm concentric gap without implant (FIG. 1). B-E: Implants coated with different amount of VEGF B: Expected daily release of 100 ng VEGF/day C: Expected daily release of 500 ng VEGF/day D: Expected daily release of 1000 ng VEGF/day. E: Expected daily release of 2000 ng VEGF/day F: Allograft. G: Expected daily release of 500ng VEGF/day - coated on hydroxyapatite.

FIG. 5: Graph of the statistics when analyzing the BV/TV from each group. * p<0.05. There is no difference between the VEGF coated implants compared to the gold standard of allograft. The group with coated hydroxyapatite has more bone-like structure and a higher BV/TV within the gap of 2 cm (FIG. 1B), which includes both the hydroxyapatite and newly formed bone. Note that the BV/TV of the implant with an expected daily release of 500 ng VEGF/day coated on hydroxyapatite includes both unresolved hydroxyapatite and newly formed bone

The present invention will now be described in more detail in the following.

### DETAILED DESCRIPTION OF THE INVENTION

Definitions Prior to discussing the present invention in further details, the following terms and conventions will first be defined:
Poly(lactic) Acid In the present context, the term “poly(lactic acid)” or “polylactic acid” or “polylactide” (PLA) is a biodegradable and bioactive thermoplastic aliphatic polyester. Polymerization of a racemic mixture of L- and D-lactides usually leads to the synthesis of poly-DL-lactide (PDLLA), which is amorphous. In a preferred embodiment of the present invention, the “poly(lactic acid)” is poly-DL-lactide (PDLLA). In the example section, PDLLA has been used.

### Vascular Endothelial Growth Factor (VEGF),

In the present context, the term “vascular endothelial growth factor”, or “VEGF” or “vascular permeability factor” (VPF) refers to a signal protein produced by cells that is believed to stimulate formation of blood vessels. The VEGF family comprises in mammals five members: VEGF-A, placenta growth factor (PGF), VEGF-B, VEGF-C and VEGF-D. VEGF-A is often just called VEGF.

In the present study, VEGF is added to Bovine Serum Albumin (BSA) (in a ratio of 1:50 (by weight)). It is believed that BSA will prevent low level binding of the aliquoted growth factor/cytokine to the storage container and prevent inactivation, while under frozen conditions. Albumin is a natural carrier protein for many growth factors in the circulation. For a purified growth factor or cytokine it will prevent precipitation of the pure protein in a watery solution, as well as sticking to the carrier vessel by hydrophobic interactions.

### Method for Coating a Medical Implant

As outlined above, the present invention relates to a novel coating composition suitable for coating a medical implant, such as for improving bone ingrowth around the medical implant. Thus, an aspect of the invention relates to a method for coating a medical implant (or part of a medical implant), the method comprising
a) providing a medical implant;
b) providing a liquid (coating) composition comprising
   0.01-0.2 mg/μl PLA, preferably poly(DL-lactic) acid (PDLLA);
   0.1-10 ng/μl VEGF;
   30-70% (by volume) chloroform;
   20-50% (by volume) organic solvent, preferably an alcohol, more preferably ethanol; preferably the organic solvent being different from chloroform; and
   2-10% water.
c) coating said medical implant (or part of the medical implant to be coated) in vitro with the composition of step b);
d) drying said coated medical implant; and
e) optionally, repeating step c) to d) at least one time.

The medical implants are implants where it would be beneficial to stimulate e.g. 30 bone ingrowth around the implant. Thus, in an embodiment, said medical implant is selected from the group consisting of a screw, a joint, a fastening mean, a bone filler, a fracture fixation device and an endoprosthetic device. In a related embodiment, the fracture-fixation device is selected from the group consisting of a plate, a screw, a nail, a pin, a wire, a thread, an arthroplasty and a cage. In yet an embodiment, the implant has a sandblasted surface.

In another embodiment the medical implant is selected from the group consisting of
Fixation devices such as screws, k-wires, nails, implants and plates;
Joint prosthesis;
Vertebral cages; and
Biomaterials, bone filler and bone grafts.

The medical implant may comprise or consist of different materials. Thus, in another embodiment, said medical implant comprises or consists of metal, preferably titanium, steel or thantalum, pure magnesium and combinations with alloys, plastic, Hydroxyapatite (HA), elastomers, acrylic resins, ceramics including TCP and other natural and synthetic polymers. In the example section titanium and Hydroxyapatite (HA) have been tested.

Different types of PLA may find use in the coating composition according to the invention. Thus, in an embodiment, the PLA is selected from the group consisting of poly(D-lactic) acid, poly(L-lactic) acid, poly(DL-lactic) acid, Poly(lactic acid) (PLA), such as poly(L-lactic 25 acid), such aspoly(DL-lactic acid), such as 20 polycaprolactone, such as poly(glycolic acid) (PGA), such as polyanhydride, for example poly(alkylene succinates), such as poly(hydroxy butyrate) (PHB), for example poly(butylene diglycolate), such as poly(.epsilon.-caprolactone) and copolymers or blends thereof, preferably poly(DL-lactic) acid. In the example section poly(DL-lactic) acid has been used.

The amount of poly(DL-lactic) acid (or another PLA) may vary. Thus, in an embodiment the liquid composition comprises in the range 0.01-0.2 mg/μl poly(DL-lactic) acid (PDLLA), preferably in the range 0.05-0.1, more preferably in the range 0.06-0.08 mg/μl.

The amount of VEGF may also vary. Thus, in an embodiment, the liquid composition comprises in the range 0.01-10 ng/μl VEGF (without BSA carrier) preferably in the range 0.02-4 ng/μl, more preferably in the range 0.2-2.5 ng/μl.

Different types of VEGF may also be used. Thus, in an embodiment, the VEGF is selected from the group consisting of VEGFA, VEGFB, VEGFC, VEGFD and PIGF1,2, preferably the VEGF is VEGFA, more preferably recombinant human VEGF165 (rVEGF165) (a member of VEGFA). In the example section the VEGFA, recombinant human VEGF165 (rVEGF165), has been used.

The amount of chloroform may also vary in the coating composition. Thus, in an embodiment, the liquid composition comprises in the range 40-70% (by volume) chloroform, such as 50-70%, or such as 55-65%, preferably 57-62% chloroform.

The amount of organic solvent may also vary in the coating composition. Thus, in an embodiment, the liquid composition comprises in the range 30-50% (by volume) organic solvent, such as 30-40%, preferably in the range 32-38%. In yet an embodiment, the organic solvent is an alcohol, preferably of the formula CₙH₂ₙ₊₁OH, where n is 1-20, more preferably n is 1-5, such as 1-3, or such as 2, most preferably the alcohol is ethanol. In the example section, ethanol has been tested.

The amount of water (aqua dest.) may also vary in the composition. Thus, in an embodiment, the liquid composition comprises in the range 2-8% water (by volume), preferably 3-7% water.

The bone ingrowth may be further improved by the addition of one or more further components. Thus, in yet an embodiment, the liquid (coating) composition further comprises one or more components selected from the group consisting of platelet derived growth factor (PDGF) AA, PDGF BB; insulin-like growth factors-1 (IGF-I), IGF-II, acidic fibroblast growth factor (FGF) (all 22 members of the FGF family .FGF1-FGF22), basic FGF, beta.-endothelial cell growth factor, FGF 4, FGF 5, FGF 6, FGF 7, FGF 8, and FGF 9; Ang1, Ang2; Matrix metalloproteinase (MMP);Semaphorins (SEMA), SEMA3; Delta-like ligand 4 (D114); transforming growth factor TGF-P1, TGF .beta.1.2, TGF-.beta.2, TGF-.beta.3, TGF-.beta.5; bone morphogenic protein (BMP) 1, BMP 2, BMP 3, BMP 4, BMP 7, 15 vascular endothelial growth factor (VEGF), placenta growth factor; epidermal growth factor (EGF), amphiregulin, betacellulin, heparin binding EGF, interleukins (IL) -1, IL-2, IL-3, IL-4, IL-5, IL-6, IL-7, IL-8, IL-9, IL-10, IL-11, IL-12, IL-13, IL-14, IL-15-18, colony stimulating factor (CSF)-G, CSF-GM, CSF-M, erythropoietin; nerve growth factor (NGF), ciliary neurotropic factor, stem cell factor, hepatocyte growth factor, modificed RNA (mRNA) cells for secretion, calcitonine gen related peptid (CGRP), Hypoxia induced factor 1 (HIF-lalpha) and platelet derived growth factor (PDGF).

However, since the coating composition only comprising one growth factor, namely VEGF, it may not be required to add further growth factors or other stimulating factors to the composition. Thus, in another embodiment, the liquid (coating) composition is free from further components selected from the group consisting of platelet derived growth factor (PDGF) AA, PDGF BB; insulin-like growth factors-1 (IGF-I), IGF-II, acidic fibroblast growth factor (FGF) (all 22 members of the FGF family .FGF1-FGF22), basic FGF, beta.-endothelial cell growth factor, FGF 4, FGF 5, FGF 6, FGF 7, FGF 8, and FGF 9; Ang1, Ang2; Matrix metalloproteinase (MMP);Semaphorins (SEMA), SEMA3; Delta-like ligand 4 (D114); 15 transforming growth factor TGF-P1, TGF .beta.1.2, TGF-.beta.2, TGF-.beta.3, TGF-.beta.5; bone morphogenic protein (BMP) 1, BMP 2, BMP 3, BMP 4, BMP 7, 15 vascular endothelial growth factor (VEGF), placenta growth factor; epidermal growth factor (EGF), amphiregulin, betacellulin, heparin binding EGF, interleukins (IL) -1, IL-2, IL-3, IL-4, IL-5, IL-6, IL-7, IL-8, IL-9, IL-10, IL-11, IL-12, IL-13, IL-14, IL-15-18 ; colony stimulating factor (CSF)-G, CSF-GM, CSF-M, erythropoietin; nerve growth factor (NGF), ciliary neurotropic factor, stem cell factor, hepatocyte growth factor , modificed RNA (mRNA) cells for secretion,calcitonine gen related peptid (CGRP), Hypoxia induced factor 1 (HIF-lalpha) and platelet derived growth factor (PDGF). In the example section, efficient bone ingrowth is obtained using only VEGF as growth factor.

Thus, in a preferred embodiment of the invention, the coating composition comprises
0.01-0.2 mg/μl poly(DL-lactic) acid (PDLLA);
0.1-10 ng/μl) VEGFA;
50-70% (by volume) chloroform;
30-40% (by volume) alcohol, most preferably ethanol; and
3-7% water.

The effect of the VEGF may be improved if a carrier is added. Thus, in yet an embodiment, the liquid composition further comprises a carrier, preferably BSA, preferably in a ratio of VEGFA to BSA in the range 1:10 to 1:100 (by weight), such as 1:30 to 1:70, or such as 1:40 to 1:60, such as 1:50. Without being bound by theory, the effect of BSA may also have an effect on the storage mechanism as BSA is a carrier/filler protein that will prevent low-level binding of the aliquoted growth factor/cytokine to the storage container and prevent inactivation, while under frozen conditions. The skilled person may find use of other relevant carriers/fillers than BSA. Thus, in an embodiment the protein carrier/filler is selected from the group consisting of bovine serum albumin (BSA), Keyhole

Limpet Hemocyanin (KLH), Concholepas concholepas hemocyanin (CCH), carrier proteins developed from Hemocaynin, melaimide and thyroglobulin and combinations thereof.

The step of coating the medical implant with the coating composition can take place in different ways. Thus, in an embodiment, said coating step c) is performed by dipping/submerging the medical implant in the liquid composition one or more times or by spraying the liquid composition onto the medical implant. In yet an embodiment, said dipping/submersion takes place for 3 seconds to 1 minute at 0-20° C., such as 3 seconds to 30 seconds minutes or such as 3 seconds to 10 seconds at 0-10° C. In another embodiment, said drying step d) is air drying, such as for 10 seconds to 5 minutes at 20-30° C., such as 20 seconds to 3 minutes or such as 30 seconds to 90 seconds at 20-30° C. In example 1, the coating method is described in further details.

In yet another embodiment, said repeating step e) takes place 1-5 times, preferably 1-3 times and more preferably 1 time or 2 times. In example 1, step e) was repeated one time.

When a coating composition is going to be used on medical implants, the composition of course has to be sterile. Thus in an embodiment, said provided medical implant is sterile. Also the coating composition is preferably sterile.

It would be an advantage if the coated medical implants could be stored for a certain period before use. Thus, in an embodiment, the obtained coated medical implant can be stored for at least 30 days at -20° C. before use as a medical implant, such as at least 60 days, such as at least 90 days, or such as 1-100 days, or such as 10-60 days.

The pH of the coating composition may vary. Thus, in an embodiment, the liquid composition has a pH in the range 3.5-8.

The volume of coating composition applicable to a medical implant of course depends on the size of the implant (or size of the part of the medical implant to 10 be coated). Thus, in an embodiment said coating composition is applied in step c) with an amount in the range 0.1 - 10 μl per mm² of surface area of the medical implant to be coated, such as in the range 0.2 - 2 μl per mm², or such as in the range 0.3 - 1 μl per mm², preferably in the range 0.4 - 0.8 μl per mm² of surface area of the medical implant to be coated. In the example section the surface of the implant was coated with a total coating of 0.6-0.7 μl per mm².

As also outlined above, and shown in the example section, different advantages have been identified for the medical implant according to the invention. Thus, in a further embodiment, the obtained medical implant is for improving bone formation and/or implant fixation and/or bone ingrowth in vivo (compared to uncoated implants, or implants coated with alternative coatings).

### Coating Composition

As described above, the invention also relates to a novel coating composition. Thus, in yet an aspect the invention relates to a liquid coating composition comprising
0.01-0.2 mg/μl PLA, preferably poly(DL-lactic) acid (PDLLA);
0.1-5 ng/μl VEGF;
30-70% (by volume) chloroform;
20-50% (by volume) organic solvent different from chloroform, preferably an alcohol, more preferably ethanol;
preferably a carrier, more preferably BSA; and
2-10% (by volume) water.

In an embodiment, liquid coating composition is for coating a medical implant (or part of a medical implant), such as for improving bone formation and/or implant fixation in vivo.

### Use of the Liquid Coating Composition

A further aspect of the invention relates to the use of a liquid coating composition according to the invention, for coating a medical implant (or part of a medical implant).

### Coated Medical Implants

In yet a further aspect, the invention relates to a medical implant coated on the surface with poly(DL-lactic) acid and VEGF.

In a preferred embodiment, said medical implant is selected from the group consisting of a screw, a joint, a fastening mean, a bone filler, a fracture fixation and an endoprosthetic device;

and/or

said medical implant comprises or consists of metal, preferably titanium, steel or thantalum, pure magnesium and combinations with alloys, plastic, Hydroxyapatite (HA), elastomers, acrylic resins, and ceramics including TCP.

In yet a preferred embodiment, said medical implant is selected from the group consisting of a screw, a joint, a fastening mean, a bone filler, a fracture fixation device and an endoprosthetic device.

In yet a further preferred embodiment, said medical implant comprises or consists of metal, preferably titanium, steel or thantalum, pure magnesium and combinations with alloys, plastic, Hydroxyapatite (HA), elastomers, acrylic resins, and ceramics including TCP.

Another preferred embodiment, said medical implant comprises or consists of metal, preferably titanium.

In an embodiment, the medical implant is coated with an amount of VEGF in the range 0.5 ng-300 ng per mm² of implant intended to be coated, such as in the range 5-200 ng per mm² of implant to be coated, such as in the range 25-120 ng per mm² of implant to be coated.

In yet an embodiment, the coated medical implant has a storage time at −20° C. for at 24 hours, such as at 7 days, such as at least 30 days, such as at least 60 days, or such as at least 90 days. Experiments have shown that there is no difference between implants used after 24 hours at −20° C. and implants stored 90 days at −20° C. (data not shown).

In a related aspect, the invention relates to a medical implant obtained/obtainable by a coating method according to the invention.

### Kit of Parts

It may be advantageous to be able to provide a kit, which can mixed before use to form the coating composition according to the invention. Thus, an aspect of the invention relates to a kit of parts comprising
A first container comprising PLA, preferably poly(DL-lactic) acid (PDLLA);
A second container comprising VEGF;
A third container comprising chloroform;
A fourth container comprising an organic solvent different from chloroform, preferably an alcohol, more preferably ethanol;
optionally, a fifth container comprising water;
optionally, a sixth container comprising a carrier, preferably BSA; and
optionally, instructions for preparing a liquid coating composition according to the invention and/or performing a method according to the invention.

In an embodiment, the kit further comprises one or more implants to be coated.

In yet a further aspect the invention relates to the use of a kit according to the invention, for coating a medical implant (or part of a medical implant). Preferably, said medical implant is selected from the group consisting of a screw, a joint, a fastening mean, a bone filler, a fracture fixation and an endoprosthetic device; and/or said medical implant comprises or consists of metal, preferably titanium, steel or thantalum, pure magnesium and combinations with alloys, plastic, Hydroxyapatite (HA), elastomers, acrylic resins, and ceramics including TCP.

### Medical Uses of Implants

If the medical implant as such is biodegradable, it may be considered a medicament. Thus, in a further aspect the invention relates to a medical implant according to the invention, for use as a medicament, with the proviso that the medical implant is biodegradable. In yet another aspect the invention relates to the medical implant according to the invention, for use as bone implant, with the proviso that the medical implant is biodegradable.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1

### Method of Coating an Implant—Materials and Methods

### Coating Procedure

The coating of the implants had the purpose of delaying the release of vascular endothelial growth factor (VEGF) from the implant. The procedure was done in a sterile environment. All equipment was sterilized and bench, gloves, and mouth 30 band were used due to the sterile procedure. The coating consisted of biodegradable PLA, Poly(D,L-Lactide) (PDLLA) combined with the carrier solution of 60% chloroform (by volume), 5% water (by volume) and 35% ethanol (by volume). The surface of the implant was coated with a total coating of 0.6-0.7μl per mm2.

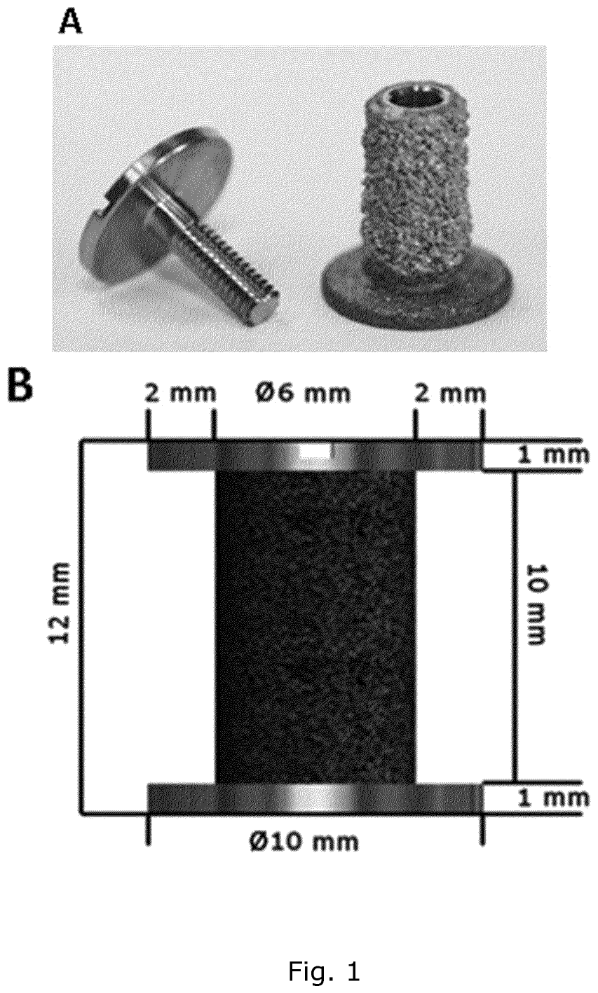

The implants were titanium implants (see also FIG. 1A) or hydroxyapatite. An ideal biomaterial should bear three basic characteristics: osteoinductive, osteoconductive, and osteogenic properties. Autografts carry all three characteristics and has been the gold standard graft material. However, harvesting autograft commonly from iliac crest might be associated with increased blood loss, wound complications, local sensory deficits, and persistent donor site pain. Allografts from donors have often been used in revision surgery, and considered as surrogate gold standard second next to autograft and carry mainly osteoconductive properties. However, beyond the question of efficacy, the potential risk of disease transmission is the biggest concern associated with the use of allograft bone.

The carrier material for the coating used in the present study is hydroxyapatite (HA), and is one of the bone substitutes most identical to bone available. This is to be used with the PDLLA VEGF coating in the femoral gap for a local effect.

The implants used had a sandblasted surface of 302 mm². 0.7 μl×302 mm²=total amount of ≈200-210 μl. The amount of PDLAA was 0.06-0.07 mg/μl. For 200 μl, this meant 12-15 mg PDLLA per implant.

The release of VEGF was estimated to be around 21 days, so the amount of total product in the solution should be divided by 21 to give the daily released dose. A release of 10Ong/day will add the total of 2100 ng to the solution. A Bovine serum albumin (BSA) carrier was used in the VEGF-A-165 in the ratio of 1:50 (by weight). This means a total of 2100 ng×50 =0.105 mg was added for a release of 100 ng/day.

The total dosages for a release of 100 ng/day, 500 ng/day, 1000 ng/day and 2000 ng/day VEGF were 2100 ng, 10,500 ng, 21,000 ng, and 42,000 ng combined with BSA in a ratio of 1:50, respectively.

During this procedure, the mixture and coating for each implant was made separately to secure the right dosages.

First, the liquid was prepared by pipette. Depending on the total volume in the implant according to area surface, 60% of chloroform were calculated. The total amount of 200 μl×60% =120 μl pr. implant. Aqua dest (water) 200 μl×5% =10 μl. Ethanol 70% 200 μl×35%=70 μl.

When the liquid solution was made, PDLLA and VEGF were added in calculated amounts. The fluid was gently rotated to dilute the VEGF component in the mixture.

A pipette or a tweezer or guidewire was used to dip the implant into the mixture. When the implant surface had been covered it was placed on a sterile table. After 30-90 seconds (depending on the surface) the coating was dry. The same dipping procedure was repeated, and it took between 30-90 seconds until the implant was dry. The implant was stored in a sterile bag at −20° C. The titanium implants used in this design is illustrated in FIG. 1.

### Animals

The sheep breed Texas/Gotland wool mixed was used. Their mean age was 4-7 years and their mean body weight was 71.0±8.7 kg. The sheep were housed in outdoor paddocks and were fed hay and compound feed throughout the experiment. The animals were housed indoors at the central animal facility 1 week prior to surgery and 2-3 days postoperation. All institutional and national and international guidelines such as ARRIVE for the care and use of laboratory animals were followed, and the Danish Animal Experiments Inspectorate approved the study.

### Surgical Procedure

As premedication, the animals received 0.2 mg/kg of Rompun. Anaesthesia was induced with 3 mg/kg of propofol 10 mg/mL, while the surgical procedures were performed under general anaesthesia (2.0% isoflurane). Under aseptic 25 conditions, and after iodine disinfection of the lateral femur, the periosteal surface was exposed by an incision through the skin. To prevent any thermal damage of the bone and surrounding tissue, a low-speed drill created a 12-mm deep cylindrical hole with a circumference of 10 mm. To remove residual bone particles, the gap was rinsed with saline before insertion of the implants forming a gap of 2 mm. The implant was placed correctly and fixated in the critical size defect. If allograft should be applied in the defect, the gap was filled with sterilized allograft. Finally, the wound was sutured in three layers. The procedure was repeated for the medial side as well as the opposite femoral condyles bilaterally. Postoperative analgesia 0.03 ml/mg Temgesic and ampicillin 250 mg/mL was administered daily 35 for 3-4 days. After 12 weeks of observation, the sheep were euthanized with an overdose of pentobarbital and both distal femurs were harvested and divided prior to further processing according to former works.

### Preparation of Specimen

The bone implant specimens were sawed orthogonally into two parts with an Exakt diamond band saw. After removal of the top washer, a bone-implant sample of 3.5 mm was prepared and stored at −20° C. until it was scanned using the microCT at 6p voxel size. Due to the preservation of the implant, only one sample was scanned at a time. The remaining part of the implanted specimen, 5.5 mm, was prepared for histological and histomorphometrical investigations. Some of those samples were still in dehydration in ethanol series (70-90%) at room temperature and embedded in methyl methacrylate. Using the vertical sectioning method using a microtome and counterstained with toluidine blue 0 to visualize mineralized bone.

### Allografts

The allograft bone was gathered from healthy sheep. The trabecular bone structure from the femur bone was divided by a manual bone mill during sterile procedure. The allografts were stored according to protocol in a freezer at −80° C.

The method Used from Schmidmaier et al:

In the protocol of Schmidmaier et al., PDLLA and chloroform was used according to their protocol, combined with VEGF in the same dosages as the present invention.

Schmidmaier et al. used a 1mm diameter K-wire, 3.5 cm in length. The surface of a cylinder is then calculated by 2 x pi x radius x length with the total surface of 109.95mm2.

For 10 k-wires they used the total of 100 mg PDLLA and 1.5 ml chloroform. This means that they used 66.67 mg PDLLA pr. 1 ml chloroform in their solution. If dividing these numbers for 1 K wire, it gives a total of 10 mg and 0.15 ml for 109.95 mm².

The implants used in the present examples had a surface area of 376 mm². The difference in the surface area is 376 mm²/109.95=3.41. The dosages-ratio is then 1:3.41 when translating their method on k-wires to the implant model according to surface area.

Then the amount that was needed to be used on the implants was calculated, when knowing the ratio is 3.41. 10mg×3.41=34.1 mg PDLLA and 0.15 ml×3.41 =0.51 ml=510 μl chloroform. This gives the double amount of volume/mixture as used in the presented examples for each implant. This mixture of PDLLA and chloroform according to the method in Schmidmaier et al. were combined with the 3 total dosages amount of 2100 ng, 10,500 ng and 21,000 ng of VEGF/BSA as used in the present method.

### Example 2

### Implants in Sheep

### Aim of Study

The present example aimed at verifying the efficiency of the implants in a sheep model.

### Materials and Methods

See example 1.

### Results

MicroCT and Histology:

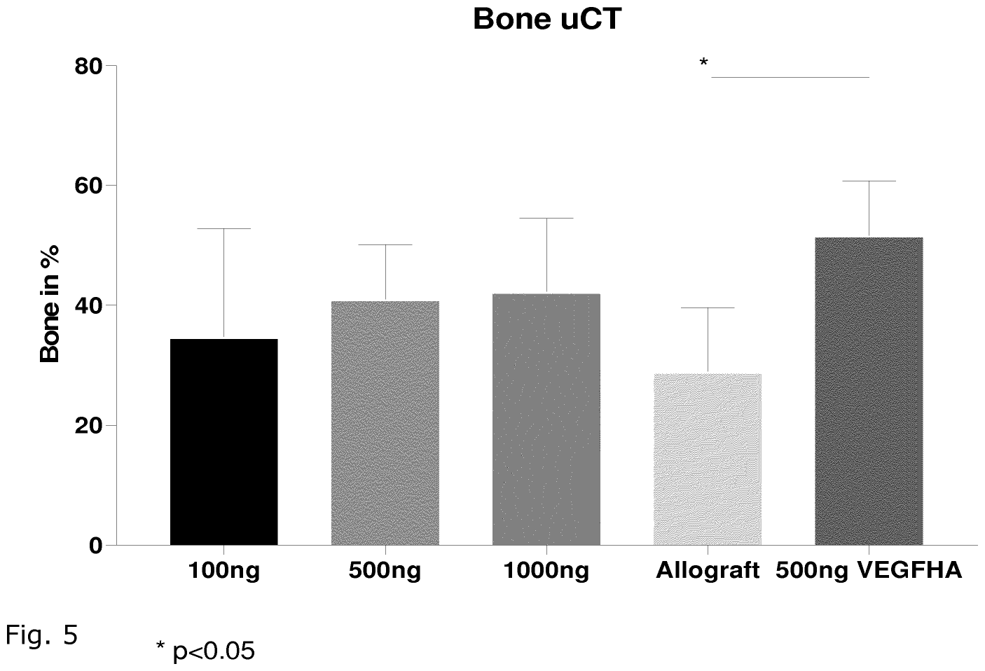

By evaluation of the top 3.5 mm of the implants (FIG. 1), the microCT scans 20 showed the similar amount of bone volume (BV)/tissue volume (TV) compared to allograft within the gap that is measured from the distance between the implant and the existing host bone in the critical size defect (FIG. 5). When compared to the group with an estimated release rate of 500ng VEGF/day were coated on the hydroxyapatite (HA) an increased BV/TV within the gap 25 compared to allograft was seen (p<0.05). This suggests that the coating composition has the same or better osteogenic and angiogenic properties than the control group of allograft.

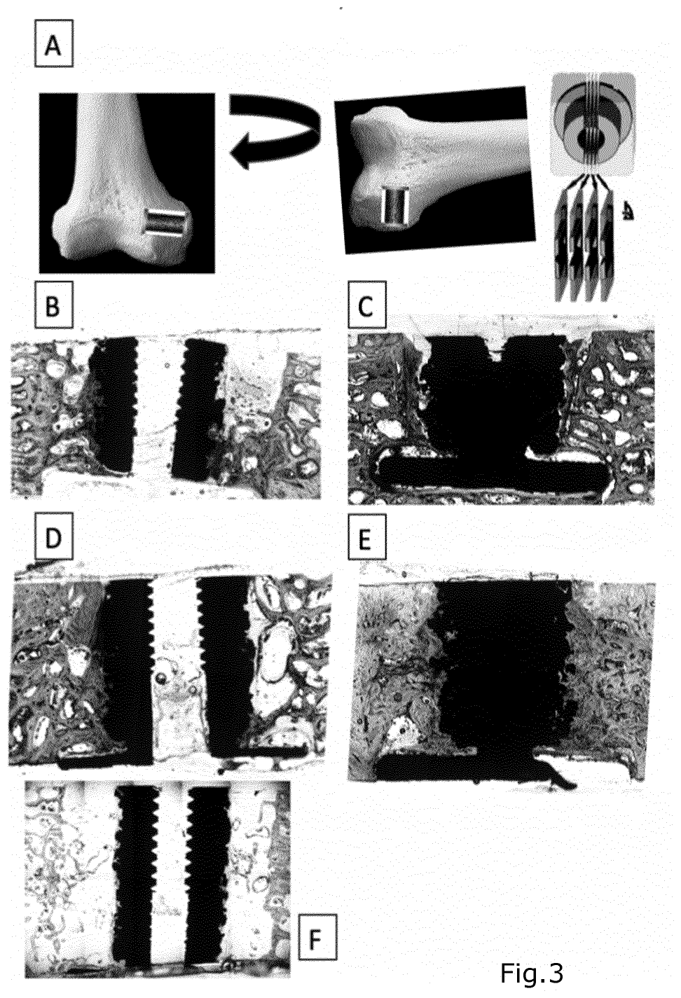

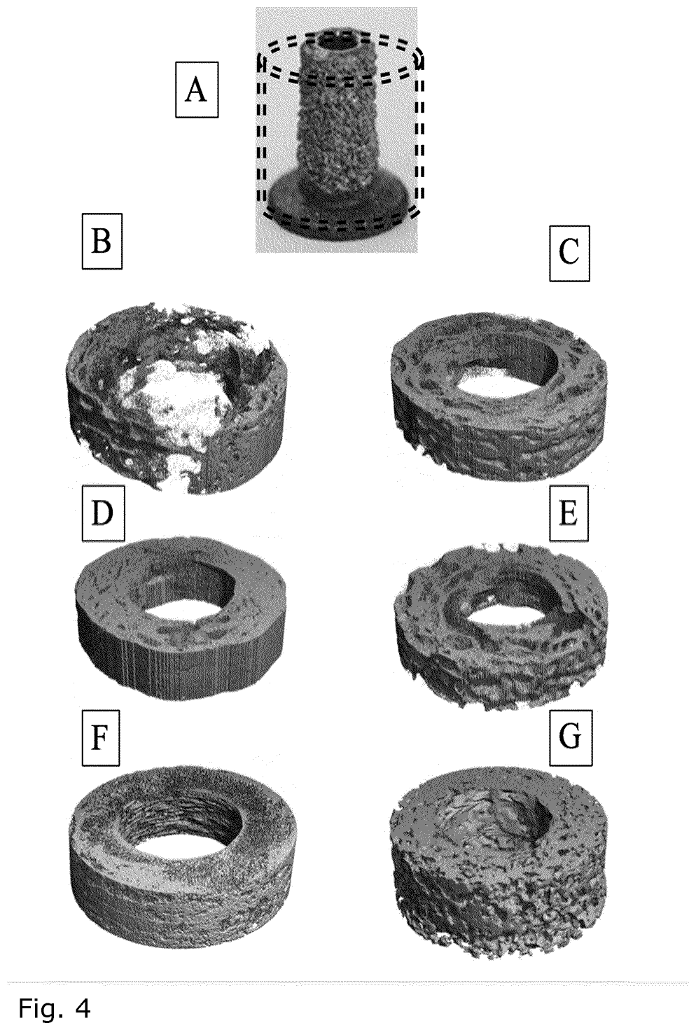

Histological images showed that the bone ingrowth to the implant is optimal when it has the right dosages, especially around the dosage of 1000 ng VEGF/day (estimated release rate) the gap had a lot of newly formed bone with good ingrowth. This is illustrated both on the microCT and the histology that the newly formed bone is very compact, and fills up the gap with ingrowth into the porous surface of implant (FIG. 4), but no significant difference to allograft. The visuals, however, give very promising results regarding the bone formation and ingrowth into implants. When an implant without a coating (coating composition according to the invention) is inserted for an observation time of 12 weeks, there is not any bone detectable in the gap (FIG. 3F).

### Conclusion

The results show that the coating composition according to the invention performs at least equally well as an allograft in relation to bone ingrowth. As previously mentioned there is a need for alternatives to allografts. When compared to a control without a coating there was a significant increase in bone ingrowth and regeneration.

When compared to the PDLLA VEGF coating on hydroxyapatite on microCT a significant higher BV/TV compared to allograft was seen.

In sum, coatings on both metal and on hydroxyapatite successfully stimulated bone regeneration

### Example 3

### Comparison to Schmidmaier et al

Aim of study

The present example aimed at comparing the coating composition disclosed on Schmidmaier et al. to the coating composition according to the present invention.

### Materials and Methods

See example 1.

### Results

It was not possible to make a detailed analysis of the results, due to the non-existing bone growth within the gap of the implant. When a laboratory technician tried to make the sections, the implant fell out if the defect, making further analysis impossible.

The results in the coating procedure showed a bad dilution of the growth factor into the composition of only PDLAA and chloroform.

### Conclusion

When using VEGF in the coating mentioned in Schmidmaier et al., no bone growth within the implant could be measured. This indicates that the method cannot contain the same growth factor without the containment and right dosages of the PDLAA and chloroform with the effect of ethanol and water for bone growth.

### Example 4

### Optimization of Liquid Composition—pH

### Aim of Study

The present example aimed at optimizing the components of the liquid composition.

A pH value was measured by an electronic pH device when using the coating composition described by Schmidmaier et al. (coating compositions 3-4) and a coating composition according to the present invention (coating compositions 1-2). The dosages corresponds to amounts that would be used for 1 implant in the femoral gap model in a 200 pl solution.

Compositions of the present invention (volume: 1mI) (Amount for 5 implants with 376 mm² surface area):

| | Coating composition 1 | Coating composition 2 |
|---|---|---|
| PDLAA | 0.06 mg/μl | 0.06 mg/μl |
| Chloroform | 60% (v/v) | 60% (v/v) |
| Water | 5% (v/v) | 5% (v/v) |
| Ethanol | 35% (v/v) | 35% (v/v) |
| VEGF | | 0.3 μg |
| BSA | | 14.7 μg |
| pH | ≈4 | ≈3.8 |
| Total volume | 1 ml | 1 ml |

Compositions modified from Schmidmaier et al (volume: 2.55 ml), (Amount for 5 implants with 376 mm² surface area):

| | Coating composition 3 | Coating composition 4 |
|---|---|---|
| PDLLA | 0.0134 mg/μl = 170.5 | 0.0134 mg/μl = 170.5 |
| | mg | mg |
| Chloroform | 100% (v/v) | 100% (v/v) |
| VEGF | | 0.3 μg |
| BSA | | 14.7 μg |
| pH | ≈3 | ≈4 |
| Total volume | 2.55 ml | 2.55 ml |

### Results

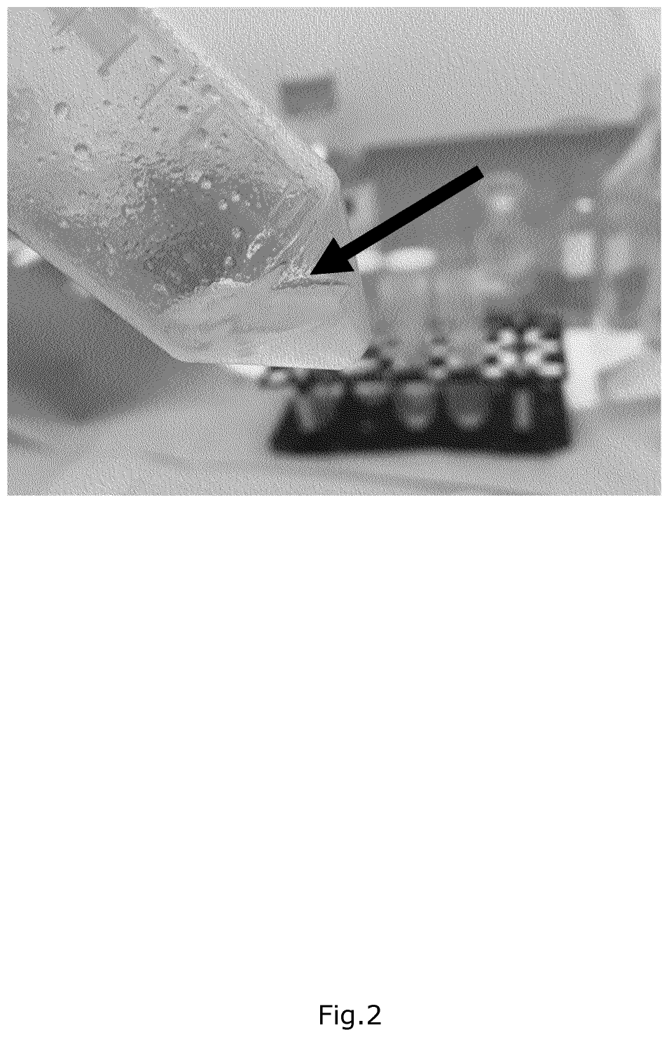

When adding VEGF to coating composition 1, thereby getting coating composition 2, the pH value increased with the addition of VEGF. On the other hand, when adding VEGF to coating composition 3, thereby getting coating composition 4, the pH value increased with the addition of VEGF. Furthermore, it was not possible to dilute VEGF in coating composition 3 (FIG. 2).

### Conclusion

The above results indicate that the reaction to these components is different whether using the method described in Schmidmaier et al., or the composition according to the present invention. This could be due to sensitivity or whether the product of VEGF has the possibility to be diluted in the solution without any ethanol or water, as shown difficult in FIG. 2.

Thus, the presence of water and an alcohol (ethanol) appears essential for getting a proper coating composition. Thus, it is not possible to simply shift the growth factors disclosed in Schmidmaier et al. with VEGF to reach a functional coating composition (see also example 6). The results of Schmidmaier et al. method had no bone formation or ingrowth when applied in the femoral gap model in sheep. Apparently, the Schmidmaier et al. method is not feasible in this critical sized defect implant model.

### Example 5

### Optimization of Liquid Composition

### Aim of Study

The present example aimed at evaluating the coating composition without VEGF.

### Results

Implants were coated with the coating composition 5 (see below) (volume: 200 μl per implant), implanted and evaluated as described in example 1.

| | Coating composition 5 |
|---|---|
| PDLLA | 0.06 mg/μl = 12 mg |
| Chloroform | 60% (v/v) |
| Ethanol | 35% (v/v) |
| water | 5% (v/v) |
| Total volume | 200 μl |

### Conclusion

When evaluating implants coated with composition 5, the implants were so loose that they could not be sectioned for scan or histology as also seen when using the method of Schmidmaier et al. combined with VEGF (Example 3). The ingrowth of bone to implant was non-existing. Thus, in the absence of VEGF, no bone ingrowth could be seen (data not shown).

### Example 6

### Optimization of Liquid Composition

### Aim of Study

The present example aimed at evaluating the coating composition disclosed in Schmidmaier et al. with different concentrations of VEGF as growth factor. Implants were coated with coating compositions 6-9, implanted and evaluated as described in example 1 (volume: 0.51 ml per 376 mm² implant surface).

| Coating | | | | |
|---|---|---|---|---|
| composition | 6 | 7 | 8 | 9 |
| PDLLA | 34.1 mg | 34.1 mg | 34.1 mg | 34.1 mg |
| Chloroform | 100% | 100% | 100% | 100% |
| | (v/v) | (v/v) | (v/v) | (v/v) |
| VEGF | 42 ng | 210 ng | 420 ng | 840 ng |
| BSA | 2058 ng | 10.290 ng | 20.580 ng | 41.160 ng |
| Equivalent | 100 | 500 | 1000 | 2000 |
| | ng/day | ng/day | ng/day | ng/day |
| pH | ≈4 | ≈4 | ≈4 | ≈4 |
| Total volume | 0.51 ml | 0.51 ml | 0.51 ml | 0.51 ml |

### Conclusion

When evaluating implants coated with coating compositions 6-9, the implants were so loose that they could not be sectioned for scan or histology. The ingrowth of bone to implant was non-existing. Thus, in the absence of ethanol and water no bone ingrowth could be seen (data not shown).

### SUMMARY OF RESULTS

The statistics of the microCT scan showed that the coating composition of the present invention has the same capability to form bone, as the current clinical gold standard of allograft. The histology showed bone ingrowth into every implant no matter the dosage of VEGF. Furthermore, when hydroxyapatite was coated with VEGF in an amount estimated to release 500 ng/day, the scans indicated more bone-like structure of HA and newly formed bone (BV/TV) within the 2 mm gap than allografts. This gives an indication of possible usage instead of allograft in critical size defects.

The coating is designed to be used on every orthopaedic implants, plates or arthroplasty to enhance both ingrowth and perhaps inhibit colonization of S. aureus due to the content of PDLLA.

When compared to the method Schmidmaier et al. with VEGF and there was no bone ingrowth in the gap region and the implants could not get sectioned for analysis. Without VEGF in the coating, no bone was regenerated. Based on the current investigation, the coating compositions of the present invention is significantly better than that of Schmidmaier et al. and this conclusion is supported by:
i. The coating composition of the present invention is considered to perform better due to the VEGF, the alcohol (ethanol), the water and e.g. also the carrier (BSA).
ii. Positive results in bone regeneration and implant fixation.

1. A method for coating a medical implant, the method comprising:
a) providing a medical implant;
b) providing a liquid composition comprising:
0.01-0.2 mg/μl PLA;
0.1-10 ng/μlVEGF;
30-70% by volume chloroform;
20-50% by volume organic solvent different from chloroform;
and
2-10% by volume water;
c) coating said medical implant in vitro with the composition of step b);
d) drying said coated medical implant; and
e) optionally, repeating step c) to d) at least one time;
wherein said liquid composition further comprises a carrier; and
wherein said medical implant is selected from the group consisting of a screw, a joint, a fastener fastening mean, a fracture fixation device and an endoprosthetic device;
and/or
said medical implant comprises metal, steel or thantalum, pure magnesium or combinations with alloys, plastic, Hydroxyapatite (HA), elastomers, acrylic resins, or ceramics.

2-20. (canceled)

21. The method according to claim 1, wherein the carrier is selected from the group consisting of bovine serum albumin (BSA), Keyhole Limpet Hemocyanin (KLH), Concholepas concholepas hemocyanin (CCH), carrier proteins developed from Hemocaynin, melaimide and thyroglobulin and combinations thereof.

22. The method according to claim 1, wherein the carrier is bovine serum albumin (BSA).

23. The method according to claim 1, wherein said coating step c) is performed by dipping or submerging the medical implant in the liquid composition one or more times.

24. The method according to claim 1, wherein the medical implant is coated on the surface with poly(DL-lactic) acid and VEGF.

25. The method according to claim 1, wherein said medical implant is selected from the group consisting of a screw, a joint, a fastener, a fracture fixation device and an endoprosthetic device.

26. The method according to claim 1, wherein said medical implant comprises metal, steel, thantalum, pure magnesium or combinations with alloys, plastic, Hydroxyapatite (HA), elastomers, acrylic resins, or ceramics.

27. The method according to claim 1, wherein said medical implant comprises metal.

28. The method according to claim 1, wherein said medical implant comprises titanium.

29. The method according to claim 1, wherein the PLA is poly(DL-lactic) acid.

30. The method according to claim 1, wherein the liquid composition comprises:
01-0.2 mg/μl poly(DL-lactic) acid (PDLLA);
0.1-10 ng/μl VEGFA;
50-70% by volume chloroform;
30-40% by volume ethanol; and
3-7% by volume water.

31. The method according to claim 1, wherein said coating is applied in step c) with an amount in the range 0.1-10 μl per mm² of surface area of the medical implant to be coated.

32. The method according to any claim 1, wherein the obtained medical implant is configured to improve bone formation, improve implant fixation and/or improve bone ingrowth in vivo.

33. A liquid composition comprising:
0.01-0.2 mg/μl PLA;
0.1-5 ng/μl) VEGF;
30-70% (by volume) chloroform;
20-50% (by volume) organic solvent different from chloroform;
a carrier; and
2-10% (by volume) water.

34. The liquid coating composition according to claim 33, wherein said coating composition is applied to a medical implant.

35. A method of using the liquid coating composition according to claim 33, to coat a medical implant, comprising
contacting the medical implant with the liquid composition of claim 33, wherein said medical implant is selected from the group consisting of a screw, a joint, a fastener, a fracture fixation device and an endoprosthetic device
or
said medical implant comprises metal, steel, thantalum, pure magnesium or combinations with alloys, plastic, Hydroxyapatite (HA), elastomers, acrylic resins, or ceramics.

36. A medical implant having poly(DL-lactic) acid and VEGF coated on the surface ,
wherein said medical implant is selected from the group consisting of a screw, a joint, a fastener, a fracture fixation device and an endoprosthetic device
or
said medical implant comprises metal, steel, thantalum, pure magnesium or combinations with alloys, plastic, Hydroxyapatite (HA), elastomers, acrylic resins, or ceramics.

37. The medical implant according to claim 36, wherein said medical implant is selected from the group consisting of a screw, a joint, a fastener, a fracture fixation device and an endoprosthetic device.

38. The medical implant according to claim 36, wherein said medical implant comprises metal, steel, thantalum, pure magnesium or combinations with alloys, plastic, Hydroxyapatite (HA), elastomers, acrylic resins, or ceramics.

39. The medical implant according claim 36, wherein said medical implant comprises metal.
